(19) United States
(12) Patent Application Publication
(10) Pub. No.: US 2011/0186052 A1
(43) Pub. Date: Aug. 4, 2011

(54) Cleaning assembly for an endotracheal tube

(76) Inventor: Orlando Morejon, Key Largo, FL (US)

(21) Appl. No.: 12/658,035

(22) Filed: Feb. 1, 2010

(51) Int. Cl. A61M 16/00

(52) U.S. Cl. 128/207.14

(57) ABSTRACT

An assembly structure to facilitate the cleaning of an interior endotracheal tube including an elongated tubular member dimensioned to pass within and along the length of the endotracheal tube. A cleaning assembly is connected adjacent the distal end of the tubular member and is structured to be disposed in an expanded position and a non-expanded position, wherein the expanded position defines a cleaning orientation of the cleaning assembly relative to the interior surfaces of the endotracheal tube. The distal end of the tubular member includes an enlarged tip portion disposed on and at least partially defining the distal end, wherein the exterior surface of the enlarged tip peripherally includes a curved and/or at least partially domed configuration structured to assume a protective orientation relative to the cleaning assembly at least when the cleaning assembly is in the non-expanded position.

### BACKGROUND OF THE INVENTION

1. Field of the Invention

This invention is directed to an assembly structured to clean and interior of an endotracheal tube and includes an elongated tubular member having an expandable cleaning assembly attached hereto. The tubular member includes an enlarged tip disposed on and at least partially defining a distal portion of the tubular member, wherein the enlarged tip is disposed, dimensioned and configured to assume a protective orientation relative to the cleaning assembly at least when the cleaning assembly is not in an expanded or cleaning orientation.

2. Description of the Related Art

Many patients in a hospital and in particular, patients in an Intensive Care Unit (“ICU”) must be fitted with endotracheal tubes to facilitate their respiration. Specifically, an endotracheal tube is an elongate, semi-rigid lumen which is inserted into a patient's nose or throat and projects down into airflow communication with the patient's respiratory system. As such, the patient either directly, or with the aid of a respiratory unit, is able to breathe more effectively through the endotracheal tube.

Recent studies have determined, however, that the accumulation of dried tracheo-bronchial secretions on the interior wall surface of an operating endotracheal tube effectively decreases the lumen cross section, and thereby significantly increases the work of breathing for the intubated patient. Moreover, increasing the work of breathing for the patient necessitates that a higher level of support be provided to compensate and often results in the patient's incubation period and ICU stay being significantly prolonged. Furthermore, it is also seen that thick secretions on the walls of the endotracheal tube often serve as a nidus for continued infection in the lungs, leading to added morbidity and hospital costs for the intubated patient.

To date, the only effective means of eliminating the accumulated secretions within an endotracheal tube has been to exchange the contaminated endotracheal tube for a new tube. However, there are several disadvantages to this procedure, such as temporary arrest of ventilatory support and the risk of complete loss of airway control. For example, re-incubation may be exceedingly difficult in patients with swelling of the soft tissue of the neck, and in patients having cervical spine immobilization. More specifically, upon removal of the endotracheal tube, the appropriate internal passages tend to close up or be otherwise difficult to isolate for reintroduction of a new endotracheal tube. Further, re-intubation of a patient can result in additional trauma to the oral, laryngeal and tracheal tissues.

Short of replacing the endotracheal tube, other procedures currently in use for maintaining a clean endotracheal tube include the use of flexible suction/irrigation catheters. Specifically, a suction/irrigation catheter is passed down the endotracheal tube and upper airways in an attempt to evacuate contaminants from the lumen. Unfortunately, although the suction/irrigation catheters generally clear the airway of watery secretions, they are ineffective at clearing the inspissated secretions that have accumulated on the inner wall surface of the endotracheal tube over the course of days. In essence, the use of a suction/irrigation catheter merely delays the inevitable, namely, that the endotracheal tube be removed and replaced.

One somewhat recent attempt to address the problems associated with the maintenance of endotracheal tubes is seen to provide a two part assembly which is introduced into the flow through passage of the endotracheal tube. Specifically, a thin interior, solid segment having a plurality of retracting bristles and a sealing gasket at an end thereof is contained within an exterior lumen. In use, the entire coupled assembly is introduced into the endotracheal tube, and an interior segment is pushed through an outer tube so that the bristles expand to engage the wall surface. In addition, a gasket member, such as a foam cylinder or balloon, expands to completely seal off the area behind the bristles. The entire device, including the upwardly angled bristles is then pulled upwardly with the gasket element completely sealing off the tube there below so that any debris removed by the bristles is retained.

Such a device, however, does not provide for the indication of an accurate insertion in order to prevent over-insertion into the endotracheal tube. Furthermore, it is seen from the need to include the bristles, the direct engagement of a gasket type member, such as the balloon, with the interior wall surface of the endotracheal tube, does not provide for the complete and effective removal of secretions, due primarily to the smooth exterior surface of the gasket. Moreover, the smooth resilient material surface also results in substantial friction between the rubbery gasket and the plastic wall surface, thereby making it quite difficult to smoothly and effectively pull the cleansing device from the endotracheal tube. Additionally, it is recognized that the upwardly angled bristle members are susceptible to complete or partial retraction as they encounter obstacles in an attempt to scrape clean the interior of the endotracheal tube. In fact, the bristle members are often quite sharp and may be damaging to the endotracheal tube or to a patient if inadvertently projected beyond the open interior, distal end of the endotracheal tube allowing for the possibility of the outwardly projecting bristle members becoming stuck outside the endotracheal tube. Also, because of the collapsing configuration of bristles, gaps will naturally exist between adjacent bristles resulting in some areas of the tube not being engaged. Accordingly, as secretions begin to build up beneath the bristles, their collapse is further restricted. Further, such a single function device necessitates that additional items be introduced into the tube, generally resulting in additional trauma to the patient, such as when suction becomes necessary.

As such, there is still a substantial need in this art for a cleaning assembly that can be used to clear endotracheal tube secretions effectively, and on a regular basis, thereby expediting ventilatory weaning and extubation of ICU patients. Further, there is a need for an effective endotracheal tube cleaning apparatus which can be easily and effectively introduced into the endotracheal tube. Such a cleaning assembly should be easily removed while effectively removing solid secretion buildup, due at least in part to its friction minimizing engagement with the interior wall surface of the endotracheal tube and/or because of its alleviation of negative pressure/suction within the endotracheal tube upon removal thereof. Additionally, there is a need for a cleaning assembly which can be accurately extended into the endotracheal tube without substantial risk of over introduction.

In addition to the referenced needs in the industry, it is also noted that an effective cleaning assembly should preferably be structured to facilitate the operative use of cooperative devices which facilitate the cleaning procedure. Moreover, the cleaning assembly should maintain maximum sterile integrity as to those components which will be used to provide an effective cleaning procedure.

### SUMMARY OF THE INVENTION

The present invention is directed towards an endotracheal tube cleaning assembly to be used to clean the interior of an endotracheal tube while it is being used in an intubated patient. Typically, the endotracheal tube is of the type that includes a central lumen, defined by an interior wall structure that extends from a distal end to a proximal end of the tube. Specifically, the endotracheal tube cleaning apparatus includes an elongate tubular member having a diameter, or transverse dimension, smaller than lumen or interior diameter of the endotracheal tube. Further, the elongate tubular member includes a distal end that is structured to be introduced and extend into the lumen of the endotracheal tube.

Also, connected to and/or disposed in overlying relation to at least a portion of the elongate tubular member is a cleaning assembly. In at least one embodiment, the cleaning assembly is disposed in adjacent but spaced relation to the distal end of the elongate tubular member. The cleaning assembly includes an inflatable or expandable bladder having an exterior cleaning surface, such as an exterior abrasive surface. The exterior cleaning surface is structured to affirmatively engage the interior wall surface of the endotracheal tube with some outward cleaning pressure, for subsequent cleaning of the endotracheal tube upon withdrawal or reciprocating movement of the elongate tubular member relative to endotracheal tube. Furthermore, in one embodiment, the irregular configuration of the exterior cleaning surface may be discontinued at an intermediate portion of the inflatable bladder such that the inflatable bladder forms a generally fluid impervious seal with the interior of the endotracheal tube. As a result, any secretions that may slip past the irregular configuration will generally not move past the fluid impervious seal and will be effectively withdrawn from the endotracheal tube. Moreover, the cleaning assembly is structured to provide an effective mechanism to gather samples of those secretions for subsequent testing.

In at least one embodiment, the cleaning assembly may comprise an expandable bladder and a sheath member disposed in at least partially overlying relation to the bladder. Further, the cleaning assembly may be secured to the elongate tubular member at a point opposite the distal end of the elongate tubular member. For instance, the cleaning assembly may have an attachment end which is disposed opposite to the distal end of the elongate tubular member that is introduced and extended into the lumen of the endotracheal tube. Moreover, the cleaning assembly may be secured to the elongate tubular member at the attachment end. Therefore, the sheath member may include the attachment end and therefore effectuate the attachment of the cleaning assembly to the elongate tubular member opposite to the distal end thereof.

In further embodiments of the present invention, the elongate tubular member may also be structured to include a recessed portion adjacent the distal end. This recessed portion has a smaller exterior diameter or transverse dimension than the remaining length of the elongate tubular member thereby effectively creating a space differential between the exterior of the tubular member in the recessed portion compared to the rest of the tubular member. The recessed portion may extend about the entire circumference of the tubular member, creating a circular band of recessed space, or it may comprise a discrete recessed area over only a portion of or along a side of the tubular member.

Accordingly, the various structural embodiments and modifications of the cleaning assembly may be capable of being disposed in and between an expanded position and a non-expanded position. When in the expanded position, the cleaning assembly extends radially outward from the outer surface of the tubular member so as to assume a “cleaning orientation”. Further, in order to facilitate insertion of the tubular member within the endotracheal tube and while the cleaning assembly is in its non-expanded orientation, the tubular member includes an enlarged tip disposed at or adjacent to or at least partially defining the distal end and possibly including the extremity of the distal end of the tubular member.

The enlarged tip preferably includes an at least partially domed or curved exterior configuration extending substantially about the entire circumference thereof. Such a curved or partially domed configuration will be such as to facilitate insertion of the tubular member and the cleaning assembly when in its non-expanded position. As such, the enlarged tip can be accurately described as being disposed in a “protective orientation” relative to the cleaning assembly before the cleaning assembly assumes the expanded, cleaning orientation. Such a protective orientation may be further defined by the outer peripheral portions or peripheral borders of the enlarged tip extending radially outward from a remainder of the outer surface of the tubular member into at least substantial alignment with the outer surface or portions of the cleaning assembly before it is expanded into the cleaning orientation. As used herein, the term “substantially aligned” relation of the outer peripheral border or other outer, peripheral exterior surfaces of the enlarged tip can be said to extend at least minimally beyond the outer surface of the cleaning assembly rather than being precisely flush therewith, when in its non-expanded position, and still be accurately described as being substantially aligned therewith.

As also described in greater detail hereinafter, the cleaning assembly may be mounted on the exterior of the tubular member adjacent to the distal end and as such adjacent to the aforementioned enlarged tip. However, in yet another embodiment as set forth above, a length of the tubular member substantially adjacent to the enlarged tip may have a recessed configuration extending continuously or partially about its outer surface. As such, the recessed portion of the outer surface of the tubular member may be dimensioned, disposed and configured to include all or at least a portion of the cleaning assembly therein. Therefore, the cleaning assembly, when in its non-expanded orientation is in substantial alignment with or disposed radially inward from an outer surface of a remainder of the tubular member.

The inclusion of the enlarged tip being disposed in a protective orientation with regards to the cleaning assembly as well as the cleaning assembly being disposed in a recessed portion of the tubular member serves to effectively eliminate or significantly reduce the possibility of the cleaning assembly becoming dislodged or being forced out of its intended shape or configuration due to interaction with the interior surface of the endotracheal tube and any secretions or other material disposed therein.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a side view in schematic form of a prior art endotracheal tube cleaning assembly in an operative orientation within an endotracheal tube.

FIG. 2 is an isolated, side, sectional view of an elongated tubular member and handle assembly of a conventional endotracheal tube cleaning apparatus.

FIG. 3 is an isolated view in partial cutaway and section of one preferred embodiment of the tubular member including an enlarged tip secured to and/or at least partially defining the distal end of the tubular member being disposed in a protective orientation relative to a cleaning assembly, which is in a non-expanded position within an endotracheal tube.

FIG. 4 is an isolated, side view in partial cutaway and section of the embodiment of FIG. 3, wherein the cleaning assembly is in an expanded, cleaning orientation relative to the interior of the endotracheal tube.

FIG. 5 is an isolated, side view in partial cutaway and section of yet another preferred embodiment of the cleaning assembly of the present invention comprising an enlarged tip disposed on or at least partially defining the distal end of the tubular member, wherein a cleaning assembly is disposed on or within a recessed portion of the tubular member in a non-expanded orientation.

FIG. 6 is an isolated, side view in partial cutaway and section of the embodiment of FIG. 5, wherein the cleaning assembly is disposed in its expanded, cleaning orientation.

Like reference numerals refer to like parts throughout the several views of the drawings.

### DETAILED DESCRIPTION OF THE PREFERRED EMBODIMENT

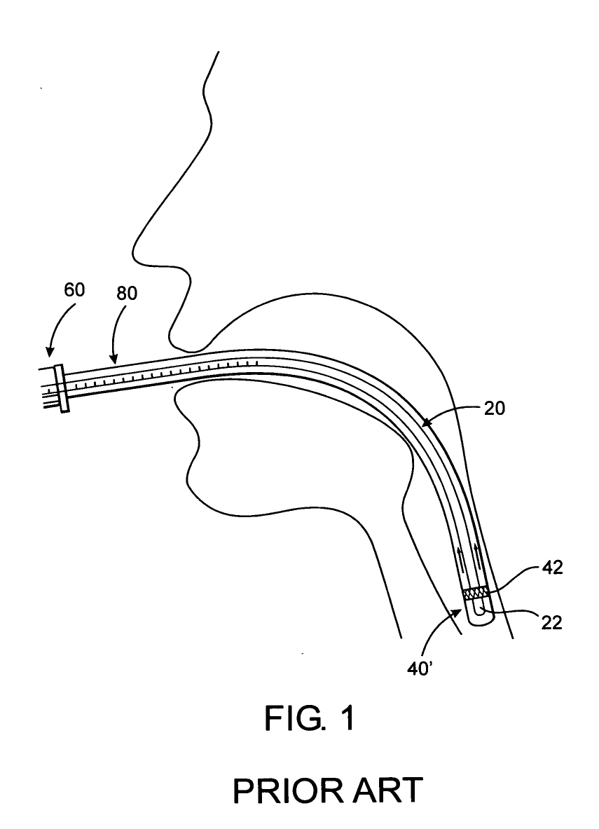

Shown throughout the Figures, the present invention is directed toward an endotracheal tube cleaning apparatus, generally indicated as 10. In particular, the endotracheal tube cleaning apparatus 10 is constructed for use with an endotracheal tube 80 that is conventionally utilized to enable a patient to breathe by insertion down the throat of a patient as schematically illustrated in FIG. 1. Generally, after prolonged periods of use, the endotracheal tube 80 will exhibit a buildup of secretions that form on the interior wall surface and can thereby obstruct airflow there through. The endotracheal tube cleaning apparatus 10 of the present invention, among other functions, is structured to facilitate the removal of those secretions in a convenient and effective manner.

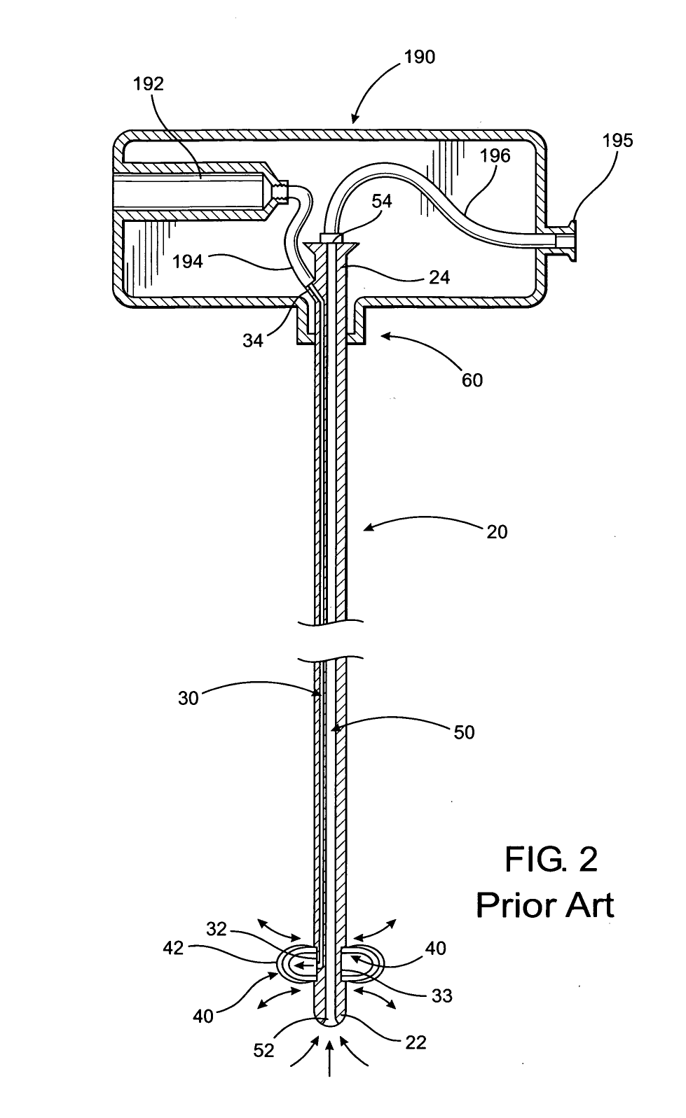

In particular, the endotracheal tube cleaning apparatus of the present includes an elongate tubular member 20 having a first/proximal end 24 (similar to that represented in FIG. 2) and a second/distal end 22. The elongate tubular member 20, which is preferably of a semi rigid construction so as to allow it to bend and conform to the operative configuration of the endotracheal tube 80 within a patient, has a length at least equivalent to a length of the endotracheal tube 80. As such, the endotracheal tube cleaning apparatus 10 can effectively reach deep down into the length of the endotracheal tube 80 for effective cleaning of even the most remotely introduced portions thereof. Furthermore, the elongate tubular member 20 is structured with a diameter smaller than the interior diameter of the endotracheal tube 80, and in fact, is preferably quite narrow so as to facilitate the introduction of the elongate tubular member 20 into endotracheal tubes of varying sizes and permit normal airflow thereabout in most circumstances.

Similar to that as represented in FIG. 2, the elongate tubular member 20 may include an inflation channel 30. Specifically, the inflation channel 30 is structured to extend from generally the first end 24 of the elongate tubular member 20 towards the second or distal end 22 of the elongate tubular member 20. Moreover, the inflation channel 30 will preferably terminate in an outlet port 32 defined generally near the second end 22 of the elongate tubular member 20. The outlet port 32 of the inflation channel 30 is structured and disposed so as to permit the escape of a fluid, such as air, there through, subsequent to its passage through the length of elongate tubular member 20 within the inflation channel 30. The outlet port 32 of the inflation channel 30 preferably extends out a side of the elongate tubular member 20, in a vicinity of the distal end 22 of the elongate tubular member 20, and may preferably extend into an annular track defined in the elongate tubular member 20.

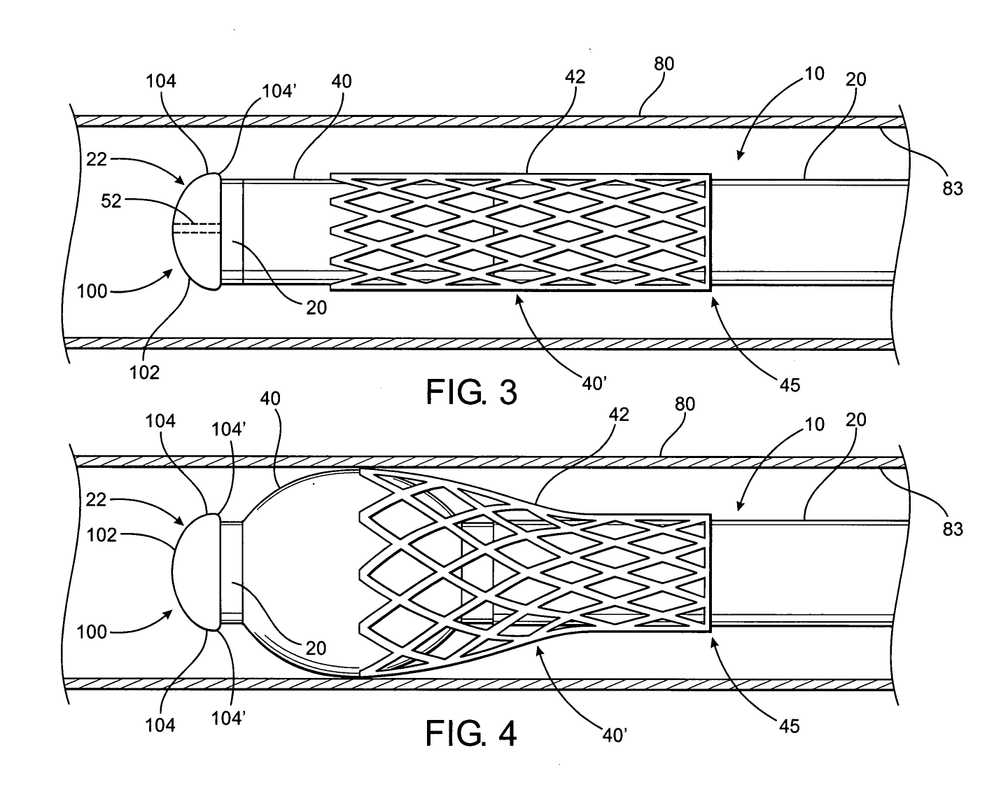

Secured to the elongate tubular member 20, also generally at the distal end 22 is a resilient or expandable material bladder 40, which is a part of the cleaning assembly 40′. Preferably the expandable bladder 40 engages the elongate tubular member 20 in communication with the outlet port 32 of the inflation channel 30. Accordingly, the resilient material bladder 40 is structured and disposed to be in fluid flow communication with the outlet port 32 and hence the inflation channel 30. Therefore, when a fluid, such as air, exits the inflation channel 30 through the outlet port 32, it will pass into the resilient material bladder 40 to result in a corresponding inflation thereof. Specifically, the resilient material bladder 40 is formed of an expandable material and is preferably structured to inflate to at least a diameter that is approximately equivalent to a diameter of the interior wall surface of the endotracheal tube 80, thereby exerting some outward pressure on the interior surfaces of the endotracheal tube 80 when it is expanded into the cleaning orientation. Additionally, the bladder 40 may be sized to be variably inflated and thereby permit effective use of the endotracheal tube cleaning apparatus 10 within endotracheal tubes 80 having varying interior diameters. The resilient material bladder 40 may be secured to the elongate tubular member 20 in a variety of fashions, as will be described in greater detail in FIG. 3 through 6, and may take on a variety of configurations to provide for appropriate inflation and secure retention at generally the distal end 22 of the elongate tubular member 20.

FIGS. 3 through 6 show additional embodiments of the present invention, wherein the cleaning assembly 40′ is secured to the elongate tubular member 20 at an attachment end 45. Specifically, in at least one preferred embodiment, the present invention comprises an elongate tubular member 20 having distal end 22 and a transverse dimension less than the lumen of the endotracheal tube 80, and a cleaning assembly 40′ disposed in overlying relation thereto having an attachment end 45 disposed in opposite relation to the second/distal end 22 of the elongate tubular member 20. In at least one embodiment, the cleaning assembly 40′ is secured to the elongate tubular member 20 at the attachment end 45. In some embodiments, the cleaning assembly 40′ may be secured to the elongate tubular member 20 exclusively at the attachment end 45.

Moreover, the cleaning assembly 40′, which overlies at least a portion of the elongate tubular member 20, is further comprised of a resilient bladder 40 and an outer periphery. This outer periphery may be formed of an exterior sheath member 42 disposed in at least partially overlying relation to the resilient bladder 40, and may be expandable. In at least one embodiment, the attachment end 45 may be formed in the exterior sheath member 42, and may be secured or attached to the elongate tubular member 20 therethrough. FIGS. 12 and 13 illustrate one example of this in which the exterior sheath member 42 is secured to the tubular member 20 at the attachment end 45 of the cleaning assembly 40′, shown in the operative, non-expanded position (FIG. 12), and in the operative cleaning positioning (FIG. 13) wherein the expanded cleaning assembly 40′ exerts a cleaning force on the endotracheal tube 80.

Attachment of the exterior sheath member 42 at the attachment end 45 prevents the sheath member 42 from becoming detached from the endotracheal tube cleaning apparatus 10 during use, such as may occur upon moving the cleaning assembly 40′ back and forth during cleaning, which may be desired if, for example, there are dried secretions that resist being broken up or removed. In addition, attachment of the exterior sheath member 42 at the attachment end 45 reduces or eliminates the possibility of “peel-back” or a rolling effect of the exterior sheath member 42 toward the second/distal end 22 of the elongate tubular member 20 during cleaning use, which would limit or decrease the effectiveness of cleaning. Accordingly, when the cleaning assembly 40′ is secured to the elongate tubular member 20, especially at the attachment end 45, the cleaning assembly 10 may be used in applications requiring greater force than if the cleaning assembly 40′ were not attached. This may be especially useful if there is a significant build-up of dried secretions, to enhance the abrasive effect, or for other situations where an increased application of cleaning force is desired.

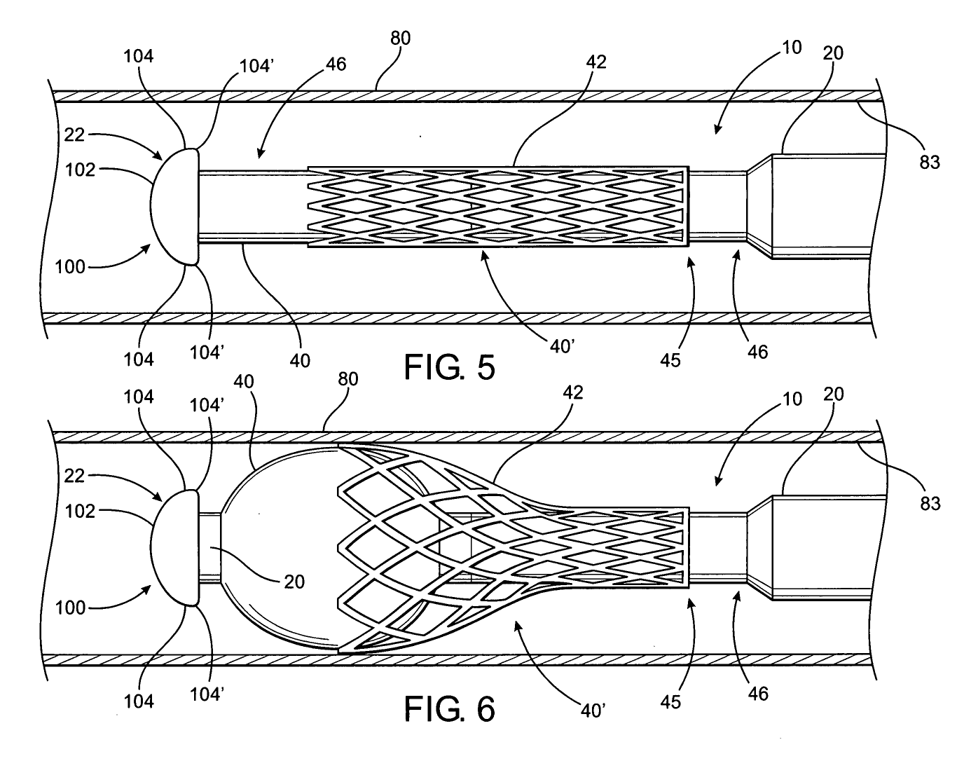

Additional embodiments of the invention include the exterior sheath member 42 attached at the attachment end 45 to the elongate tubular member 20, wherein the elongate tubular member 20 comprises a recessed portion 46. For example, the elongate tubular member 20 comprises a distal end 22, a proximal end 24, and a length defined therebetween. The recessed portion 46 comprises at least a portion of the length of the elongate tubular member 20, and in some embodiments, the recessed portion 46 is disposed proximate or near the distal end 22. This recessed portion 46 has a transverse dimension less than that of the rest of the elongate tubular member 20, such that the exterior surface within the recessed portion 46 is reduced from the exterior surface of the elongate tubular member 20. In the embodiment illustrated in FIGS. 5 and 6, this recessed portion 46 comprises the entire circumference of the tubular member 20, creating a circular band of recessed space. In another embodiment, the recessed portion 46 may comprise only a discrete portion of or is disposed along a side of the tubular member 20 (not shown). The cleaning assembly 40′ is disposed in overlying relation to at least a part of the recessed portion 46 in these embodiments, such as is depicted in FIG. 14.

With reference to FIG. 5, the cleaning assembly 40′, specifically the bladder 40 and the exterior sheath member 42, are disposed in the recessed portion 46. In one example of the operative, non-expanded position of this embodiment, the bladder 40 and exterior sheath member 42 remain entirely within the depth of the recessed portion 46. That is, the outer periphery of the cleaning assembly 40′ is structured to extend beyond the outer edge or transverse dimension of the elongate tubular member 20 when the cleaning assembly 40′ is at least partially expanded. For instance, the cleaning assembly 40′ may be expanded radially outward. Accordingly, the cleaning assembly 40′, when disposed in the recessed portion 46, may not protrude from the elongate tubular member 20 and therefore may not come in contact with the interior wall surface of the endotracheal tube 80 while placing or positioning the cleaning apparatus 10 into the operative position. This may be particularly useful when cleaning narrow endotracheal tubes, as it allows for an easier placement of the elongate tubular member 20 within the endotracheal tube 80. For instance, there is less of a risk of dislodging and/or pushing dried secretions into the patient during the placement of the elongate tubular member 20 into the operative position, prior to inflation and subsequent cleaning. Once properly placed, the bladder 40 is expanded or inflated and the cleaning apparatus 10 is now in cleaning position in which the exterior cleaning surface of the cleaning assembly 40′, and more in particular, the outer periphery or sheath member 42 contacts the endotracheal tube 80 in cleaning engagement, and the cleaning assembly 40′ exerts a cleaning force on the endotracheal tube 80 once expanded. FIG. 6 shows one example in which the cleaning assembly 40′ extends radially outward when expanded.

The cleaning assembly 40′ may be expanded or inflated by introduction of a fluid, such as air. Referring now to FIG. 2, disposed opposite the outlet port 32 of the inflation channel 30, and also connected in fluid flow communication with the inflation channel 30 is an inlet port 34. Specifically, the inlet port 34 is structured to permit the introduction of a fluid, preferably air, into the inflation channel 30 for subsequent inflation of the resilient material bladder 40. While this inlet port 34 may be positioned anywhere in the elongate tubular member 20, it is preferred that it be positioned generally near the proximal end 24 thereof (see FIG. 2) in order to permit the facilitated introduction of fluid there through when the elongate tubular member 20 is substantially introduced into the endotracheal tube 80.

As further represented in FIGS. 3-6, one preferred embodiment of the present invention comprises structuring of the tubular member 20 to include an enlarged tip portion generally indicated as 100 connected to and/or at least partially defining the distal end 22 of the tubular member 20. More specifically, the enlarged tip 100, as represented in FIGS. 3 and 4, is disposed immediately adjacent to the cleaning assembly 40′ including an expandable and/or inflatable bladder 40 and an exterior sheath member 42. Similarly, with reference to the embodiment of FIGS. 5 and 6, the cleaning assembly 40′ is connected to or disposed at least partially within the recessed portion 46 of the tubular member 20. As such, the enlarged tip 100 is connected to and/or at least partially defines the extremity of the distal end 22 of the tubular member 20 in the manner represented.

Specific structural features of the enlarged tip 100 includes an exterior, exposed, peripheral surface 102 preferably having an at least partially domed or curved configuration extending over all or at least a majority thereof. Additional structural features of the enlarged tip 100 include a substantially continuous peripheral border 104 extending in surrounding relation to the innermost end of the exterior dome shaped surface 102. Further, the continuous peripheral border 104 comprises a substantially curved or at least partially beveled configuration, as at 104′, extending along the continuous length of the peripheral border 104, wherein the curve 104′ is transversely oriented to the length of the border 104, as represented in FIGS. 3-6. The provision of the curved configuration 104′ minimizes trauma and eliminates or significantly reduces the possibility of the enlarged tip 100 becoming “hooked” on the interior 83 of the endotracheal tube 80, especially as the tubular member ids withdrawn from the interior of the endotracheal tube. The disposition, dimension and configuration of the enlarged tip 100, including the peripheral border 104,104′ also facilitates the penetration of the tubular member 20 through any type of secretive material, including blood clots, mucus plugs, etc., as the tubular member is advanced through the endotracheal tube 100.

Accordingly, the dimension and configuration of the enlarged tip 100 of the tubular member 20 is such as to be disposed in a “protective orientation” relative to the cleaning assembly 40′ whether it is mounted on or connected to the exterior of the tubular member 20 as in the embodiments of FIGS. 3 and 4 or connected to or mounted at least partially within the recessed portion 46 in the embodiments of FIGS. 5 and 6.

The aforementioned “protective orientation” may be additionally described and defined as at least a portion of the exterior peripheral, curved surface 102, such as at the peripheral border 104,104′ extending transversely or radially outward from the remainder of the exterior surface of the tubular member 20 into substantial alignment with the exterior of the cleaning assembly 40′ such as with the exterior of the sheath 42. The term “substantial alignment” is meant to describe the peripheral border 104 or other portions of the exterior, curved surface of the enlarged tip extending being at least in a substantially flush orientation with the outer surfaces of the cleaning assembly such as the bladder 40 and/or sheath 42. Alternatively, the dimension and configuration of the enlarged tip 100 may be such that the “substantial alignment” with the cleaning assembly 40′ includes at least a portion thereof such as, but not limited to, the peripheral border 104 extending radially beyond the outer surface of the cleaning assembly 40′ including either the bladder 40 and/or the sheet 42, when the cleaning assembly is not in its expanded orientation and therefore prior to it assuming its cleaning orientation as represented in FIGS. 4 and 6.

Further, when in the cleaning orientation, the bladder 40 will be expanded and/or inflated causing the sheath member 42 to expand outwardly into its cleaning orientation relative to the interior surfaces of the endotracheal tube 80. When so disposed, the cleaning assembly 40′ will preferably extend radially outward beyond the exterior surface 102 including the peripheral border 104 so as to effectively assume the aforementioned cleaning orientation.

At least one structural modification of the enlarged tip 100 is evident in a comparison of the embodiments of FIGS. 3 and 5. More specifically, in the embodiment of FIG. 3, the enlarged tip 100 includes an opening 52 corresponding to the suction inlet or opening 52 as represented in FIG. 2. As such, the opening or inlet passage 52 is disposed in fluid communication with interior portions such as the channel 50 also represented in FIG. 2 wherein the secretions or other material on the interior of the endotracheal tube 80 may be removed by suction. In contrast, the embodiment of FIG. 5 includes a closed, enlarged tip 100 absent any type of opening or in flow channel 52 as represented in FIG. 3.

Accordingly, the protective orientation of the enlarged tip 100, relative to the cleaning assembly 40′, facilitates the insertion of the tubular member 20 in a manner which protects and prevents or substantially reduces the possibility of the various components of the cleaning assembly 40′ coming into contact with, being dislodged or otherwise disfigured when engaging the interior surfaces of the endotracheal tube 80 or any of the secretions or other material collected therein. As such, the curved exterior surface 102, also accurately described as having a partially domed configuration, facilitates the passage of the distal end 22′ of the tubular member 20 into the interior and along the length of the endotracheal tube 80, when the cleaning assembly 40′ is in its non-expanded position and therefore not in its cleaning orientation, as represented in the embodiments of FIGS. 3 and 5.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. An assembly structured to clean an interior of an endotracheal tube, said assembly comprising:
a) an elongated tubular member dimensioned to pass into and along the length of the endotracheal tube interior,
b) said tubular member including a proximal end and distal end,
c) a cleaning assembly connected to said tubular member in adjacent relation to said distal end,
d) said cleaning assembly structured to be expanded, radially outward from the tubular member into a cleaning orientation,
e) said distal end having an enlarged tip disposed between said cleaning assembly and an extremity of said distal end, and
f) said enlarged tip configured to assume a substantially protective orientation relative to said cleaning assembly.

2. An assembly as recited in claim 1 wherein said protective orientation comprises said enlarged tip extending radially outward from at least a portion of said tubular member corresponding to said cleaning assembly.

3. An assembly as recited in claim 1 wherein said cleaning assembly is disposable into and between an expanded position and a non-expanded position; said expanded position defining said cleaning orientation.

4. An assembly as recited in claim 3 wherein said protective orientation further comprises said enlarged tip at least partially extending radially outward from a remainder of said tubular member into at least substantially aligned relation to said cleaning assembly, when said cleaning is in said non-expanded position.

5. An assembly as recited in claim 4 wherein said enlarged tip is configured to define a peripheral portion thereof disposed in substantially flush relation to a cleaning surface of said cleaning assembly at least when said cleaning assembly is in said non-expanded position.

6. An assembly as recited in claim 1 wherein said enlarged tip includes an exterior surface comprising a curved configuration extending over at least the majority thereof.

7. An assembly as recited in claim 1 wherein said enlarged tip includes an exterior surface comprising an at least partially domed configuration extending over the majority of said enlarged tip.

8. An assembly as recited in claim 7 wherein said partially domed configuration and said enlarged tip further comprise a peripheral border having a curved configuration extending along a length of said peripheral border, said peripheral border disposed adjacent said cleaning assembly.

9. An assembly as recited in claim 8 wherein said peripheral border is disposed in outwardly extending relation from a remainder of said tubular member in substantially aligned relation with a cleaning surface of said cleaning assembly, when said cleaning assembly is in said non-expanded position.

10. An assembly as recited in claim 7 wherein said at least partially domed configuration further comprises a closed configuration.

11. An assembly as recited in claim 7 wherein said at least partially domed configuration comprises at least one aperture formed in said enlarged tip, said aperture disposed in fluid communication with an interior of said tubular member.

12. An assembly as recited in claim 1 wherein said tubular comprises an exterior recessed portion; said cleaning assembly at least partially disposed within said recessed portion at least when said cleaning assembly is in said non-expanded position.

13. An assembly as recited in claim 12 wherein said recessed portion comprises an elongated configuration and a lesser transverse dimension that at least the majority of a length of said tubular member.

14. An assembly as recited in claim 12 wherein said enlarged tip is disposed adjacent a distal end of said recessed portion and includes a peripheral portion disposed in substantially aligned relation with said cleaning assembly when in said non-expanded position.

15. An assembly as recited in claim 14 wherein enlarged tip includes an exterior surface comprising an at least partially domed configuration extending over the majority of said enlarged tip.

16. An assembly structured to clean an interior of an endotracheal tube, said assembly comprising:
a) an elongated tubular member dimensioned to pass into and along the length of the endotracheal tube interior,
b) a cleaning assembly connected to said tubular member in adjacent relation to a distal end of said tubular member,
c) said cleaning assembly disposable in and between an expanded position and a non-expanded position; said expanded position defining a cleaning orientation,
d) an enlarged tip disposed on said tubular member on an at least partially defining said distal end thereof,
e) said enlarged tip including an exterior surface comprising a curved configuration extending over at least the majority thereof, and
f) said curved configuration of said exterior surface disposed and dimensioned to define a protective orientation of said enlarged tip relative to said cleaning assembly at least when said cleaning assembly is in said non-expanded position.

17. An assembly as recited in claim 16 wherein said protective orientation further comprises enlarged tip at least partially disposed in substantially aligned relation to said cleaning assembly, when said cleaning is in said non-expanded position.

18. An assembly as recited in claim 17 wherein said curved configuration comprises an at least partially domed configuration extending over a majority of said tip portion.

19. An assembly as recited in claim 18 wherein said partially domed configuration and said enlarged tip further comprise an inner peripheral border disposed adjacent said cleaning assembly.

20. An assembly as recited in claim 18 wherein said peripheral border comprises a continuous length and a transversely curved configuration extending along said continuous length.

21. An assembly as recited in claim 18 wherein said at least partially domed configuration further comprises a closed configuration.

22. An assembly as recited in claim 18 wherein said at least partially domed configuration comprises at least one aperture formed in said enlarged tip, said aperture disposed in fluid communication with an interior of said tubular member.
